(19) United States
(12) Patent Application Publication
(10) Pub. No.: US 2020/0129709 A1
(43) Pub. Date: Apr. 30, 2020

(54) WEARABLE INSPIRATION SYNCHRONIC ULTRASONIC NEBULIZER-HUMIDIFIER DEVICE

(71) Applicant: Albert Ky, Alameda, CA (US)

(72) Inventor: Albert Ky, Alameda, CA (US)

(21) Appl. No.: 16/172,828

(22) Filed: Oct. 28, 2018

(51) Int. Cl. A61M 11/00; A61M 16/16; A61M 39/08; A61M 16/00

(52) U.S. Cl. CPC A61M 11/005; A61M 16/16; A61M 39/08; A61M 2209/088; A61M 2205/3303; A61M 2210/0618; A61M 2016/0018; A61M 16/024

(57) ABSTRACT

A wearable inspiration synchronic ultrasonic nebulizer-humidifier device is presented. The headset coupled nebulizer logic electrically connected to nebulizer controlling nebulizer crystal oscillation and breathing signal. Nebulizer housing is firmly attached to distal end of flexible tubing for flowing liquid from the liquid reservoir ampoule to the nebulizer housing containing nebulizer a piezoelectric crystal with electrical coupling to nebulizer control logic, fluid compartment, jet and aerosol nozzle for inspiration-expiration spray providing users wearing a portable humidifier-nebulizer device to adjust nostril aerosol for synchronous inhalation, nozzle position and spray angle.

### BACKGROUND

### Field of the Invention

The present invention generally relates to portable humidifier-nebulizers and specifically to wearable inspiration synchronic ultrasonic humidifier-nebulizers.

### Background

The humidifier has evolved from a bulky steam vapor producing water heater, to ultrasonic nebulizer floor-room type stand, to a desktop model, then to a hand held device.

Vaporizers in the form of a steam humidifier, warm mist humidifier, heats or boils water, releasing steam and moisture into the air. However, boiling water requires significantly more energy than other techniques. The heat source can overheat, causing the product to melt, leak, and or start fires

An impeller humidifier, cool mist humidifier, uses a rotating disc to fling water at a diffuser, which breaks the water into fine droplets that float into the air. Some designs carry a risk of spreading bacteria or mold into the air and are usually noisier than humidifiers.

An ultrasonic humidifier uses a ceramic diaphragm vibrating at an ultrasonic frequency to create water droplets that silently exit the humidifier in the form of cool fog. Usually the mist gets forced out by a tiny fan, while some ultra mini models have no fans. The models without fans are meant mainly for personal use. Ultrasonic humidifiers use a piezoelectric transducer to create a high frequency mechanical oscillation in a film of water. This forms an extremely fine mist of droplets about one micron in diameter and are quickly evaporated into the air flow.

The spray mist type humidifiers use a pipe, usually a small plastic one, to bring water directly to an electrically controlled valve, atomizer, which forces the water through a tiny orifice causing it to break up into tiny particles. In this humidifier, water mist is sprayed directly into the supply air, and the mist is carried into the premises by the air flow. Unlike the humidifiers that boil water, these water droplets will contain any impurities that are in the reservoir, any pathogens growing in the stagnant tank will also be dispersed in the air. Ultrasonic humidifiers should be cleaned regularly to prevent bacterial contamination from being spread throughout the air.

Special disposable demineralization cartridges may also reduce the amount of airborne material, but any mineral dust may have negative health effects. If the tap water is used, and it contains minerals then the ultrasonic or impeller humidifiers will produce a “white dust”, calcium most commonly, which usually spreads over furniture, and is attracted to static electricity generating devices such as CRT monitors. The white dust can be prevented by using distilled water or a demineralization cartridge in ultrasonic humidifiers. But the nature of these static humidifiers in use waste energy to humidify larger volumes of ambient air and leave moisture in and on surrounding surfaces which will need cleaning

Some problems with the current ultrasonic nebulizer handset. If the nebulizer is held proximate to the nose, a strong or high nebulizer stream will irritate the nasal mucosa and the skin surrounding the nose. Moreover, a continuous nebulizer ejection stream does not aid the user during the expiration phase and wastes power and water. As with the other humidifiers, the unused atomized stream spreads moisture and more to the air and surface, creating biological and unwanted surface effects on furniture and equipment.

There are man-made environments where the humidity is maintained by a digital control system at certain specified levels. These may be uncomfortable to some people and not others. An example of this problem exists in all airplane cabins, where the air humidity is set to a default at around 20%, an airline “company policy”, since this is not a regulated by the FAA. An overabundance of humidity will trigger the 20% sensor to dry the air. To some passengers this overly dry air in the cabin might be one of the multiple causes of discomfort or even insomnia in passengers.

There are nebulizers with aerosol masks, strapped to the users head with tube umbilical anchoring the user to fluid and power supplies. These are location constricting and very expensive. What is needed are less expensive and mobile solutions.

Another example of humid air problems comes from automobile environments in winter, where windows are closed. An over abundance of humidity will cause interior fogging of the windshield, leading to vicious cycle of: fogging, triggering causing an increase a/c heat blowing drier air, causing more nebulization and in turn causing more fogging on the windshield.

What is needed are humidifiers that don't waste energy humidifying unused air, don't spray moist air on surrounding people and surfaces, don't fog windows and obscure visibility, don't emit humidifier spray upon exhale, and follow an individual's surrounding intake air without over spray of humidified air to surroundings.

### SUMMARY

The present invention discloses a wearable inspiration synchronic ultrasonic nebulizer-humidifier device comprising a headset base holder, with a rechargeable power supply, liquid reservoir ampoule, earpiece and nebulizer control logic. The rechargeable power supply is coupled to nebulizer logic electrically coupled to nebulizer for controlling nebulizer crystal oscillation and breathing synchronous signal. Nebulizer housing is firmly attached to distal end of the headset earpiece supported flexible tubing for flowing liquid from the liquid reservoir ampoule to the distal end coupled nebulizer housing. The nebulizer housing contains nebulizer a piezoelectric crystal with electrical coupling to nebulizer control logic, fluid compartment, jet and aerosol nozzle. Breath inspiration-expiration spray synchronizing mechanism, and user interface for setting control logic parameters for synchronization, timer delay and aerosol spray volume provide users wearing a portable humidifier-nebulizer device to adjust nostril aerosol for synchronous inhalation, nozzle position and spray angle.

### BRIEF DESCRIPTION OF DRAWINGS

Specific embodiments of the invention will be described in detail with reference to the following figures.

FIG. 1 illustrates front and side views of a wearable ultrasonic nebulizer headset in an embodiment of the invention.

FIG. 2 illustrates an inspiration-expiration synchronic subsystem of a wearable ultrasonic nebulizer in an embodiment of the invention.

FIG. 3 illustrates an optimal angle for the inspiration injection nozzle of a wearable ultrasonic nebulizer in an embodiment of the invention.

FIG. 4 illustrates wearable ultrasonic nebulizer synchronizer head sets in an embodiment of the invention.

FIG. 5 is a schematic of a nebulizer compartment ultrasonic nebulizer spray head and timer aspects of an embodiment of the invention.

FIG. 6 is a schematic of a wearable ultrasonic nebulizer synchronizer logic for an aspect of an embodiment of the invention.

### DETAILED DESCRIPTION

In the following detailed description of embodiments of the invention, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

### OBJECTS AND ADVANTAGES

The present invention discloses a wearable aspiration synchronized ultrasonic nebulizer device.

It is an object of the invention to increase the efficiency of the nebulizer spray nozzle as aimed at a nostril.

It is another object of the invention to draw nebulizer stream only upon the aspiration or intake part of the respiration cycle.

It is yet another object of the invention to reduce the nebulizer stream intensity of ejection and increase the humidified stream intake from aspiration.

It is yet another object of the invention to extend the supply resources of device ported energy and water.

It is also an objective of the invention to extend the supply of any medication mixed included with a humidifier supply water reservoir.

It is yet another objective of the invention to avoid humidifying ambient surrounding air not lung bound and to reduce moisture condensing on surrounding surfaces.

It is also and objective of the invention to overcome deficiencies in the present market offerings for humidifiers.

To overcome some problems with nebulizer nozzle inefficiency, in the present invention, the plane of the nebulizer spray head or nozzle, housing a piezoelectric quartz, is angulated at 45 degree to a nostril centerline. The intensity of the nebulizer stream is reduced, such that the humidified stream nozzle velocity of ejection will just reach the nostril orifice, stream intake suction depending on aspirating to draw the nebulized air into nostril orifice.

To reduce wasted spray energy and water, the nebulizer timing circuit and mechanism is designed to spray humidifier only during the inspiration phase of the respiration cycle. The inspiration—expiration synchronic nebulization method is used and described in embodiments below.

An Embodiment of a Wearable Inspiration Synchronic Nebulizer

Human aspiration and expiration occurs in a variable rhythm due to several factors among them type of weather, humidity/dryness, physical exertion, intention, etc. A signal, initiated by expiration sensors, start the inspiration phase in the respiration cycle so that the humidifier device can create and inject the nebulized injection at the start of inhalation, not before and not too long afterward. In an average or “normal respiration pattern”, inspiration follows 2 seconds or so after the onset of the expiration. Thus inhalation onset may extend during intentional deep breathing, as in therapeutic respiration with mixture of medication; or contract duration during fast breathing. But to be sure, the time delay between exhalation will be variable and so the nebulized stream must accommodate the measured realtime cycle.

Several methods to sense cessation of the expiration and to trigger the oncoming nebulization are possible. A sensed change of CO2, a sensed change of temperature from the exhalation, or the reduction in blowing force of the expiration itself. In an embodiment of the invention the exhalation force will signal the inhalation cycle and will initiate the nebulizer to spray stream humidified air following a delay.

A wearable humidifier, in an embodiment of the invention will utilize a wearable headset, not unlike expensive hear muffs, with afixed and integrated components necessary for a wearable nebulizer to spray humidified air upon an inhalation signal all the while resting comfortably somewhere about an individual's head.

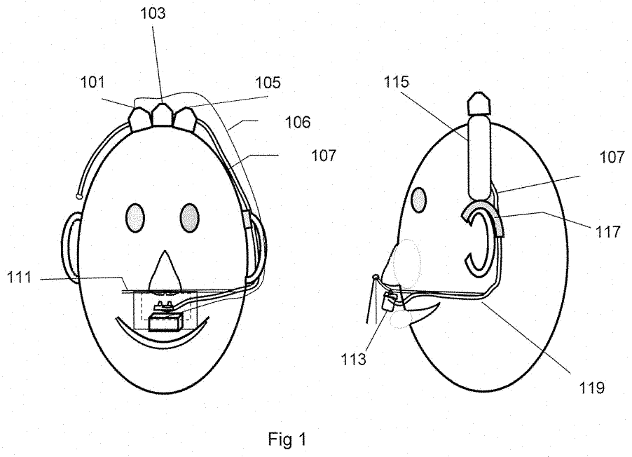

FIG. 1 illustrates front and side views of a wearable ultrasonic nebulizer device in an embodiment of the invention.

An embodiment of wearable inspiration synchronic ultrasonic nebulizer-humidifier device begins with a headset base 115 having attachment components of a rechargeable power 101 coupled to headset 115 attached nebulizer 113 and timer logic 103 with switch. Also attached to the headset 115 are a water container 105 coupled to tubing 107 119 which extend to and supply water to a nebulizer housing 113. The earpiece 117 of each side can also serve as housing for other components. From the earpiece component 117, extends a solid flexible transverse bar 111, the tubing 107 for convecting fluid from the water supply ampoule 105 to the nebulizer 113 and signal wire 106 and with timer switch, wire 106 adjacent to the tubing 107 for carrying signal to and from the nebulizer. The tubing 107 and wire 106 are coupled to the nebulizer housing 113 and inspiration triggering contact respectively. More concisely the head wearable headset base attaches a rechargeable power supply, liquid reservoir ampoule, earpiece and nebulizer control logic. The rechargeable power supply coupled to nebulizer logic which is electrically coupled to nebulizer for controlling nebulizer crystal oscillation and breathing synchronous signal. The nebulizer housing is firmly attached to distal end of the headset earpiece supported flexible tubing and the flexible tubing provides a conduit for flowing liquid from the liquid reservoir ampoule to the adjustably supporting the nebulizer housing.

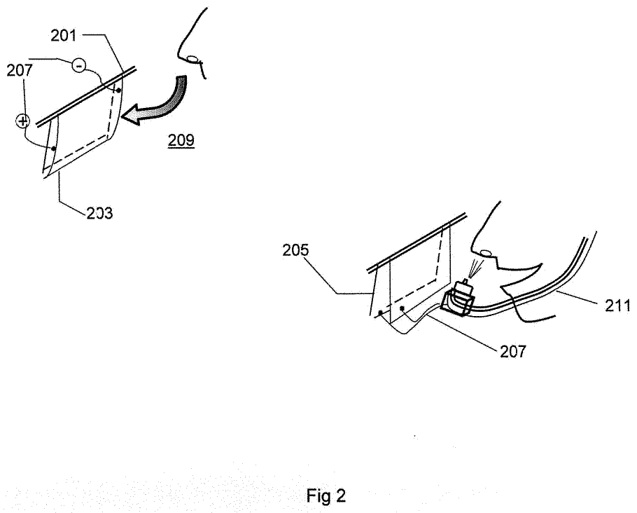

FIG. 2 illustrates an inspiration synchronic triggering subsystem of a wearable ultrasonic nebulizer in an embodiment of the invention.

In this embodiment of the synchronous triggering for the nebulizer spray The pressure sensor is an assembly of two pieces of conducting planes 203 205, shown here enlarged for illustration purposes. Conduction planes 203 205 are suspended from the transversal bar 201, each electrically connected to opposite terminal of a timer switch by conductors 207. An outside plane 205, enlarged here for illustration, can be as thick as 0.12 mm, rigidly suspended approximately at 20 degrees from a vertical. The inside plane 203 can be as thick as 0.08 mm, of flexible conductor suspended vertically, but flexibly to rotate about the bar by force of exhale. The suspended plane can also be torsion spring coupled to the transverse bar to open the following exhalation cessation. This embodiment inspiration triggering contact connected to electrical conductor 207 to timer logic. The transverse bar 201, is flexibly positioned optimally in front and below the nostrils, to receive the maximal nostril exhalant force 209 from the expiration, connecting the planes 203 205 and closing a switch 207, carrying signal 211 to timer and nebulizer logic. A time delay is set at the timer switch before the activation of the nebulizer which will last, by default, for a set delay. A physical setting to adjust the nebulizer activation time and another setting to change the triggering delay time are provided and coupled to the timer switch logic. Also, a setting for the intensity of the nebulizer current is provided, to adjust the inhalation cycle “puff” volume and quality of the nebulizer. These adjustments required for subjective user comfort to avoid irritation to the nasal mucosa of a excessive nebulizer/nozzle “puff” or ejection. Thus embodiment provides breath inspiration-expiration spray synchronizing mechanism.

In short, the tubing is coupled to a nostril proximate transverse bar switch having two flexible thin more-or-less nose perpendicular oriented electrical conducting sheets spring or angle separated, with switch close position upon nostril exhalation informing nebulizer logic

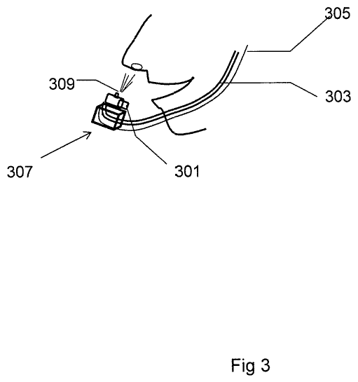

FIG. 3 illustrates an optimal inspiration nozzle angle of a wearable ultrasonic nebulizer in an embodiment of the invention.

The nebulizer compartment 307 contains the nebulizer and spray nozzle 309 as well as a pressure sensor 301 which sends signal 305 to timing logic which in turn processes any delay and response signal for fluid to be transported via fluid reservoir coupled tube 303 to source the nebulizer 307. The nebulizer spray nozzle 309 head angle is adjustable through the flexible or bendable fluid tube 303 or channel on the nebulizer compartment 307 for optimal nostril angle spray and thus not restricted to any fixed position. Thus a user wearing a portable humidifier-nebulizer device can adjust nostril aerosol for synchronous inhalation, nozzle position and spray angle. In an embodiment of the invention a pressure sensor informs the nebulizer control logic of cessation of exhalation onset of inhalation

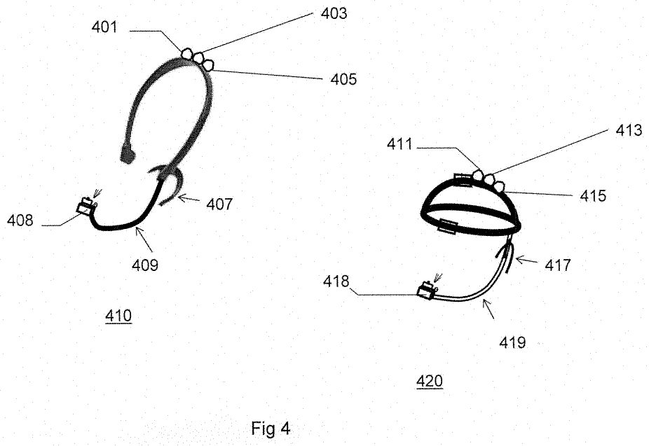

FIG. 4 illustrates wearable ultrasonic nebulizer synchronizer head sets in an embodiment of the invention.

Two wearable humidifier headsets 410 420 embodiments are shown. A flexible head set 410 of semi-rigid or bendable plastic, composite, rubber or metal of roughly head curvature for fitting on a head, inside a hat or head garment is shown. A headset coupled rechargeable power source 401 provides power to the timer switch and ultrasonic nebulizer logic 403 as well as if any pressure to fluid reservoir ampoule 405. An ear piece 407 serves to keep the headset firmly in position and also to support the tubing 409 and any wiring to-from a nebulizer component 408. An elastic head strap mounted head set 420 illustrate yet another embodiment for the wearability of a humidifier device. An elastic headset coupled rechargeable power source 411 provides power to the timer switch and ultrasonic nebulizer logic 413 as well as if any pressure to fluid reservoir ampoule 415. An ear piece 417 serves to keep the headset firmly in position and also to support the tubing 419 and any wiring to-from a nebulizer component 418. The headset base can be made from flexible plastic, composite, metal, rubber, cloth or combinations.

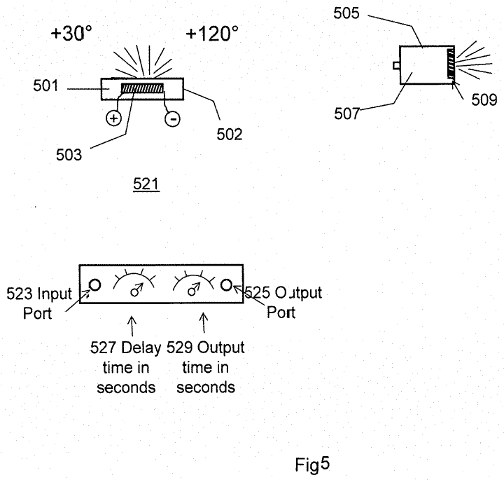

FIG. 5 is a schematic of a nebulizer compartment and ultrasonic nebulizer spray nozzle head and user settings interface aspects of an embodiment of the invention.

A nebulizer compartment 502 with immersed peizo crystal 521 will structurally house 502 a quartz or piezoelectric crystal is 503 electrically connected with power and the piezoelectric quartz crystal immersed in liquid 501 for generating entrained air bubble ejection out of the spay nozzle. Another embodiment can have an ultrasonic wave nebulizer with an electric oscillator generating a high frequency ultrasonic signal providing the mechanical vibration of the piezoelectric element vibration in contact with a liquid reservoir, crystal with sufficient vibration frequency vibration to produce a vapor mist. The nozzle spray direction can range from 30° to 120° from the nebulizer housing or compartment horizontal. The nebulizer housing contains the nebulizer crystal with electrical coupling to nebulizer control logic, fluid compartment, jet and aerosol nozzle. A user interface for setting control logic parameters for synchronization, timer delay and aerosol spray volume can is electrically coupled to the nebulizer logic.

In another embodiment nozzle compartment 505 a liquid 507 sources a piezoelectric nebulizer head 509 for adjustable angle dispersal nebulizer aerosol. A mesh/membrane is vibrated at the top of the local compartment liquid reservoir, and convects a mist of very fine aerosol through the holes. The jet nebulizer head nozzle is positioned such that the jet of aerosol is delivered mostly into the inlet of the nostril within 30° of alignment in parallel with the axial centerline of the jet outer diameter. Embodiments of the humidifier can have various nebulizer mechanisms for creating aerosol including an ultrasonic nebulizer, jet nebulizer head or vibrating mesh nebulizer.

User controls are with electrical connects 523 525 to logic for injection delay 527 and spray duration 529 are included for user adjustment of the nebulizer. The Nebulizer jet spray is hand adjustable for user nostil proximity and angle.

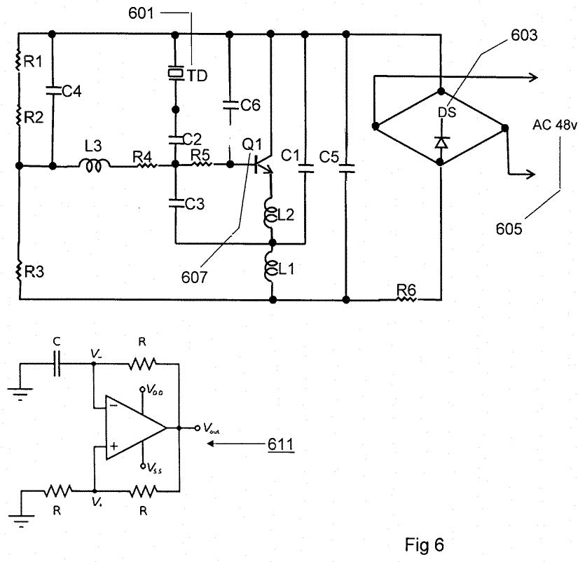

FIG. 6 is a schematic of a wearable ultrasonic nebulizer synchronizer logic in an embodiment of the invention. FIG. 6 shows nebulizer logic with a grounded collector transistor Q1 607 type self oscillation circuit. R1-R6 are resistors, C1-C6 are capacitors, L1-L3 are inductors and TD 601 is a piezo-electric vibrator for generating ultrasonic vibration.

The vibrator TD is mounted a side adjacent to water to be atomized. one of the electrodes of the vibrator is coupled with a collector of the transistor Q1 601 and other electrode contacts directly with water. Oscillation logic is supplied with D.C. voltage obtained from full wave rectifier DS 603 voltage. Other electronic logic 611 that produces a periodic oscillating electronic signal, converts the direct, DC, from a power supply to an alternating current signal is shown.

Therefore, while the invention has been described with respect to a limited number of embodiments, those skilled in the art, having benefit of this invention, will appreciate that other embodiments can be devised which do not depart from the scope of the invention as disclosed herein. Other aspects of the invention will be apparent from the following description and the appended claims.

What is claimed is:

1. A wearable inspiration synchronic ultrasonic nebulizer-humidifier device comprising:
a headset base holder,
a head wearable headset base with a rechargeable power supply, a liquid reservoir ampoule, an earpiece and nebulizer control logic;
the rechargeable power supply coupled to the nebulizer control logic;
the nebulizer control logic electrically coupled to a nebulizer for controlling a nebulizer crystal oscillation for synchronous spaying upon receipt of triggering signal from the control logic;
a nebulizer housing firmly attached to a distal end of the earpiece supported with a flexible tubing;
the flexible tubing for flowing liquid from the liquid reservoir to and adjustably supporting the nebulizer housing;
the nebulizer housing containing the nebulizer crystal with electrical coupling to the nebulizer control logic, a fluid compartment, a fluid compartment jet and an aerosol nozzle;
the nebulizer control logic providing a breath inspiration-expiration synchronizing spray, and
a user interface for setting control logic parameters for synchronization, timer delay and aerosol spray volume;
whereby user's wearing a portable humidifier-nebulizer device can adjust nostril aerosol for synchronous inhalation, nozzle position and spray angle.

2. A wearable inspiration synchronic ultrasonic nebulizer-humidifier device as in claim 1 wherein the flexible tubing is coupled to a nostril proximate transverse bar switch having two flexible thin vertical electrical conducting sheets spring or angle separated, with switch for informing nebulizer logic.

3. A wearable inspiration synchronic ultrasonic nebulizer-humidifier device as in claim 1 further comprising a pressure sensor for informing the nebulizer control logic of sensor triggering signal.

4. A wearable inspiration synchronic ultrasonic nebulizer-humidifier device of claim 1, wherein the headset base can be made from a set of materials consisting essentially of flexible plastic, composite, metal, rubber, and cloth.

5. A wearable inspiration synchronic ultrasonic nebulizer-humidifier device of claim 1, wherein the nebulizer can be from a set of nebulizers including an ultrasonic nebulizer, jet nebulizer and vibrating mesh nebulizer.

6. A method for a wearable inspiration synchronic ultrasonic nebulizer-humidifier device comprising the steps of:
providing a wearable headset;
a headset a consisting of the head wearable headset base having a rechargeable power supply, liquid reservoir ampoule, earpiece and nebulizer control logic;
recharging the power supply for providing electrical power to the nebulizer control logic;
electrically coupling the nebulizer control logic to a nebulizer for controlling nebulizer crystal oscillation for synchronous spaying upon receipt of triggering signal from the nebulizer control logic;
attaching nebulizer housing to a distal end of a headset earpiece supported with a flexible tubing;
adjustably supporting the nebulizer housing and flowing liquid via the flexible tubing from the liquid reservoir ampoule to the nebulizer housing;
nebulizer housing containing nebulizer crystal with electrical coupling to nebulizer control logic, fluid compartment, jet and aerosol nozzle;
providing headset attached user interface for control logic parameters for synchronization, timer delay and aerosol spray volume settings, and
control logic synchronizing breath inspiration-expiration with sensor
providing signal for nebulized nozzle spray to proximity of nostril
whereby wearing a portable humidifier-nebulizer device provides adjustable nostril humidified aerosol synchronous with inhalation, adjustable nozzle position and spray angle.

7. A method for a wearable inspiration synchronic ultrasonic nebulizer-humidifier device as in claim 6 wherein the flexible tubing is coupled to a nostril proximate transverse bar switch having two flexible thin vertical electrical conducting sheets spring or angle separated, exhalation closing switch and informing nebulizer logic.

8. A method for a wearable inspiration synchronic ultrasonic nebulizer-humidifier device as in claim 6 further comprising the steps of informing nebulizer control logic of cessation of exhalation onset of inhalation via the nebulizer housing mounted pressure sensor.

9. A method for a wearable inspiration synchronic ultrasonic nebulizer-humidifier device as in claim 6 wherein the headset base can be made from flexible plastic, composite, metal, rubber, and cloth.

10. A method for a wearable inspiration synchronic ultrasonic nebulizer-humidifier device as in claim 6 wherein the nebulizer can be from a set of nebulizers including an ultrasonic nebulizer, jet nebulizer and vibrating mesh nebulizer.
